(19) United States
(12) Patent Application Publication
(10) Pub. No.: US 2005/0148824 A1
(43) Pub. Date: Jul. 7, 2005

(54) Transabdominal surgery system

(76) Inventors: Dwight P. Morejohn, Davis, CA (US); Charles S. Taylor, Stockton, NJ (US); Harry Leonard Green II, Santa Cruz, CA (US)

Correspondence Address: LAW OFFICE OF ALAN W. CANNON, 834 SOUTH WOLFE ROAD, SUNNYVALE, CA, 94086, US

(21) Appl. No.: 10/748,733

(22) Filed: Dec. 30, 2003

(51) Int. Cl. A61B 1/32

(52) U.S. Cl. 600/208

(57) ABSTRACT

Systems and methods for producing a substernal space accessed via a sub-xyphoid incision. The space produced facilitates carrying out surgery, such as coronary artery bypass graft surgery with minimal trauma to a patient. Transabdominal approaches described utilize devices that lift the sternum and depress the abdomen or perform each such task as well as compress the sides of a patient's thorax, or lift the sternum and compress the sides of the thorax. Various devices including screw drives, winches, inflation and linkage mechanisms are described to achieve the stated results.

### FIELD OF THE INVENTION

This invention relates to approaches for performing surgery, especially cardiac surgery by way of creating a sub-xyphoid incision and opening a substernal working space, particularly for performing a coronary artery bypass graft (CABG) procedure.

### BACKGROUND OF THE INVENTION

Diseases of the cardiovascular system affect millions of people each year and are a leading cause of death throughout the world. The cost to society from such diseases is enormous both in terms of the number of lives lost as well as in terms of the costs associated with treating patients through traditional surgical techniques. A particularly prevalent form of cardiovascular disease is a reduction in the blood supply leading to the heart caused by arteriosclerosis or other condition that creates a restriction in blood flow at a critical point in the cardiovascular system.

Treatment of such a blockage or restriction in the blood flow leading to the heart is, in many cases, treated by a surgical procedure known as a CABG procedure, more commonly known as a “heart bypass” operation. In the CABG procedure, a surgeon “bypasses” the obstruction to restore normal blood flow to the heart either by attaching an available source vessel to the obstructed target coronary artery or by removing a portion of a vein or artery from another part of the body, to use as a graft, and installing the graft between a point on a source vessel and a point on a target artery, either one sometimes being referred to as a “host” vessel.

To restore the flow of blood to the heart, a fluid connection is established between two vessels. This is known as producing an “anastomosis.” Traditionally, a source vessel, such as a source artery with an unobstructed blood flow, e.g., the left internal mammary artery (LIMA), or a bypass-graft having one end sewn to an unobstructed blood source such as the aorta, is sewn to a target occluded coronary artery, such as the left anterior descending (LAD) artery or other vessel, that provides blood flow to the muscles of the heart.

Although CABG procedures have become relatively common, a procedure itself can be lengthy and traumatic and can damage the heart, the cardiovascular system and the central nervous system. In a conventional CABG procedure, the surgeon makes an incision down the center of the chest, cuts through the sternum, performs several other procedures necessary to attach the patient to a heart-lung bypass machine, cuts off the blood flow to the heart, and then stops the heart from beating in order to complete the bypass. The most lengthy and traumatic surgical procedures are necessary, in part, to connect the patient to a cardiopulmonary bypass (CPB) machine to continue the circulation of oxygenated blood to the rest of the body while the anastomoses are completed.

In recent years, a growing number of surgeons have begun performing CABG procedures using surgical techniques especially developed so that the CABG procedure can be performed while the heart is still beating. In such procedures, there is no need for any form of CPB support, no need to perform the extensive surgical procedures necessary to connect the patient to a cardiopulmonary bypass machine, and no need to stop the heart. Accordingly, the patient suffers less injury and requires less recovery time. Furthermore, significant expense is avoided.

Several access approaches have been attempted to facilitate CABG procedures. Instead of cracking the chest of a patient, procedures have been attempted through comparatively small incisions, typically one or two, in the chest.

However access to the trans-abdominal space of the patient is accomplished, traditionally, anastomosis completion is a particular challenge. Making a series of appropriately placed sutures through extremely small vessels on the surface of the heart while the heart muscle continues to beat requires great dexterity. In cases where the target coronary artery is temporarily obstructed, e.g., to maintain adequate visibility and avoid excessive blood loss, it is especially important that the anastomosis procedure be performed rapidly to avoid ischemic damage to the heart. Further adding to the difficulty of the procedure is the fact that the working space and visual access are often quite limited. The surgeon may be working through a small incision in the chest, for example, or may be viewing the procedure on a video monitor if the site of the surgery is viewed via surgical scope. The vessel, and particularly the arteriotomy to which a source vessel is to be anastomosed, may also be very difficult for the surgeon to see as it may be at least partially obscured by layers of fat or other tissue.

The difficulty of the beating-heart CABG procedure has been lessened by hardware adapted to stabilize the heart, particularly at the site of the anastomosis. Further improvements have been made with respect to how such stabilizing tools are mounted and also how any sutures used are retained.

Efforts are also currently underway at proving sophisticated approaches to creating the anastomosis. Many different approaches are currently be explored, many of them sutureless, which use clips, staples or other features to replace the function of the sutures. After an incision is created in a host vessel to receive blood flow from a graft, a graft/connector combination loaded into a deployment instrument is set in place, thus forming an anastomosis.

The present invention finds utility especially in connection with advanced anastomosis procedures in which graft and host vessel connections are made using tools requiring somewhat less access than produced by a full or partial sternotomy. While the present invention offers less complete access to the chest cavity of a patient, its approach is quite adequate for performing many CABG procedures. It is anticipated that this will increasingly become the case as robotic surgery technology continues to develop.

Regardless of how an anastomosis is completed, whether with the most-recently developed techniques, or by manual suture application during a stopped-heart procedure), the present invention offers a significantly less traumatic surgical approach than previously available. The present invention avoids the creation of sizable chest access ports or other incisions penetrating the rib cage.

By accessing the thoracic cavity transabdominally, pain and recovery time associated with cracked ribs, cut cartilage and bone are avoided. Accordingly, the present invention provides a significant advance in patient care. Those with skill in the art will appreciate the utility and advantages connected to the features of the invention described herein. Whatever the case, it is contemplated that some variations of the invention may only afford certain advantages, while others will present each of them.

### SUMMARY OF THE INVENTION

Features of the invention provide for thoracic cavity access, preferably, by way of a sub-xyphoid incision. An incision made through a patient's abdomen provides access that may be used in performing cardiac surgery when the incision is held open and positioned by the devices of the present invention.

In general terms, the present invention is a transabdominal access device comprising an upper and a lower separator portion, the upper and lower separator portion operatively configured to wedge or hold open an incision in a patient and form an abdominal cavity opening by depressing the abdomen at the incision while elevating the sternum of the patient. The upper separator portion of the transabdominal access device may comprise active mechanisms in the form of various screws, wratchets and/or pulleys to aid in sternal lifting. The lower separator portion may include an abdominal depressor/pusher portion, and positioning features for the abdominal depressor/pusher may also be provided with like features. The devices described may be supported against the body of a patient or by bracketing, and in particular, bracketing secured to a surgical table. Specialized separator (e.g., lifter and depressor) features are also described. Furthermore, rib compression features aiding in substernal space creation and maintenance are also disclosed. Rib compression may be achieved by an independent application of force, for instance, by pads advanced by screws, or alternately, rib compression may be coordinated with sternal lifting/retraction. Coordinated rib compression and sternal lifting by certain embodiments of the invention may be accomplished through various linkage-type setups or by at least one constrained inflatable bladder.

The present invention includes systems comprising any of these features described herein. Methodology described in association with the devices disclosed also forms part of the invention. The invention further comprises such hardware and methodology as may be used in connection with that described herein which is incorporated by reference.

### BRIEF DESCRIPTION OF THE DRAWINGS

Each of the following Figs, provides an example diagrammatically illustrating aspects of the present invention. Like elements in the various Figs, are indicated by identical numbering. For the sake of figure clarity, some such numbering is omitted.

FIG. 1A shows an oblique view of a patient with his thoracic cavity held open by one embodiment of the device according to the present invention inserted through a sub-xyphoid incision; FIG. 1B shows a side view of the situation of FIG. 1A in partial cross-section along line A-A.

FIGS. 2-4 show oblique views of various embodiments of the invention including features for adjusting the relation between elements of the invention.

FIG. 5 shows an oblique view of another embodiment of the invention including features for adjusting the relation between elements of the invention and, optionally, their orientation to a patient's body.

FIG. 6A shows an oblique view of yet another embodiment of the device of the invention including both abdominal and shoulder supports; FIG. 6B shows a top view of the device shown in FIG. 6A.

FIG. 7 shows an oblique view of another embodiment of the invention including pelvic and shoulder supports in a highly adjustable configuration.

FIG. 8 shows an oblique view of another embodiment of the invention stabilized by a pair of lockable, multi-jointed arms connected to a surgical table.

FIG. 9 shows an oblique view of an alternative embodiment of the invention affixed to a surgical table and having independently adjustable patient interface elements.

FIG. 10 shows an oblique view of another embodiment of the invention with independently adjustable lifter and depressor elements, the lifter formed, in part, by surgically-placed attachments.

FIG. 11 shows an oblique view of another independently-adjustable embodiment of the invention including an adjustable brace.

FIG. 12 shows an oblique view of yet another independently-adjustable embodiment of the invention, with separate lifter components and a full brace affixed to an operating table.

FIG. 13 shows an oblique view of lifting and depression features as shown in FIG. 12 in connection with such tools as may be used in performing a CABG procedure.

FIGS. 14 and 15 show oblique views of a patient acted upon by lifting and depression device features similar to those in the Figs. above, but with the addition of axial-acting thoracic cavity compression features to aid in substernal space expansion.

FIG. 16 shows an oblique view of a linkage-type device for thoracic lifting and compression.

FIG. 17A shows a front view of another embodiment of a linkage-type thoracic lifting and compression device; FIG. 17B shows a cross section side view taken along line B-B of a hook that may be employed by the device in FIG. 17A to effect sternal lifting; FIG. 17C shows a side view of a foot subassembly of the device shown in FIG. 17A.

FIGS. 18A-18D show a front view of a linkage configuration like that of FIG. 17A as it progressively acts upon a cross-sectional model of a patient thorax.

FIG. 19A shows an oblique view of another embodiment of a linkage-type apparatus for thoracic cavity lifting and compression further comprising a depressor-type device; FIG. 19B shows a side view of the apparatus in FIG. 19A illustrating preferred angular placement of the upper separator portion of the apparatus for thoracic cavity lifting.

FIG. 20 shows a cross-sectional view of another embodiment of the invention utilizing constrained inflatable members for lifting and compressing a patient's thorax.

### DETAILED DESCRIPTION OF THE INVENTION

Before the present invention is described in detail, it is to be understood that this invention is not limited to the particular embodiments set forth and may, of course, vary. Various changes may be made to the invention described and equivalents may be substituted without departing from the true spirit and scope of the invention. In addition, many modifications may be made to adapt to a particular situation, material, composition of matter, process, process step or steps to the objective, spirit and scope of the present invention. All such modifications are intended to be within the scope of the claims made herein. Furthermore, where a range of values is provided, it is understood that every intervening value, between the upper and lower limit of that range and any other stated or intervening value in that stated range is encompassed within the invention. That the upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications, patents and patent applications mentioned herein are incorporated herein in their entirety. The referenced items are provided solely for their disclosure prior to the filing date of the present application. Nothing herein is to be construed as an admission that the present invention is not entitled to antedate such material by virtue of prior invention.

It is also noted that as used herein and in the appended claims, the singular forms “a,” “and,” and “the” include plural referents unless the context clearly dictates otherwise. In the claims, the terms “first,” “second” and so forth are to be interpreted merely as ordinal designations, they shall not be limiting in themselves. Further, the use of exclusive terminology such as “solely,” “only” and the like in connection with the recitation of any claim element is contemplated. Further, it is contemplated that any optional feature of the inventive variation(s) described herein may be set forth and claimed independently or in combination with any one or more of the features described herein. Finally, it is contemplated that each subcombination of elements as may possibly be set forth in claims made hereto form aspects of the invention, even if not separately handled in this Detailed Description.

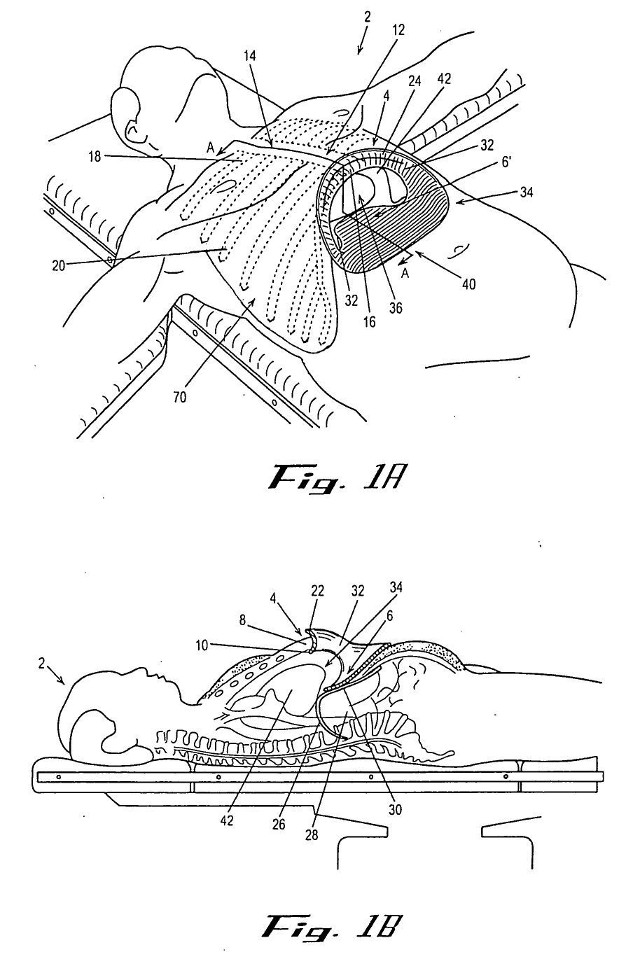

Turning now to FIG. 1A, it illustrates a very basic embodiment of the invention in connection with a human subject or patient 2. The embodiment comprises an upper spreader portion 4 and a lower spreader portion 6.

The upper spreader portion 4 includes a contact surface 8 as seen in cross-section in FIG. 1B. The upper spreader portion 4 includes a hook 22, whereby a lower portion 10 of the hook 22 lifts the lower ribcage margin 12 including the sternum 14, xyphoid process 16, costal cartilages 18 and portions of ribs 20. An upper portion of the hook 22 serves as a stop to limit advancement under the xyphoid process and/or sternum. A rail 24 for attaching various instruments as will be described further below may be included in the upper spreader portion 4.

The lower spreader portion 6 is adapted to displace the diaphragm 26 and abdominal organs such as the liver 28 downward and inferiorly. A depressor portion or element 30 of the lower spreader portion 6 includes depressor surface 31 which interfaces with the abdominal surface of the patient to press the patient's tissue downward to help form the abdominal cavity. A simple tongue-like depressor structure 30 is shown in FIG. 1A. The depressor surface 31 may be shaped concave-up as observed in cross-section in FIG. 1B, or as in other in embodiments, the depressor surface 31 is essentially flat or concave-down. The plan-form shape of the overall lower spreader portion 6 may vary as well.

In the embodiment shown in FIGS. 1A and 1B, the device comprises spreader body side members, first and a second side portions 32, which span the distance between the upper and lower spreader portions 4 and 6, respectively. Together, the two side body members and spreader portions form a shell 34 that holds open a substernal cavity 36 by lifting the sternal area/lower ribcage margin 12 and depressing the abdomen at an incision 38 preferably made below the xyphoid, yet above the diaphragm.

Shell 34 is preferably a substantially-rigid polymeric structure. Suitable materials for use include but are not limited to nylon, polycarbonate, encased carbon fiber, injection moldable plastics, shaped acrylics, thermosetting polymers, ABS polymer, or other substantially rigid, biocompatible polymers.

Each of the embodiments of the invention described below are used in a similar manner to the embodiment shown in FIGS. 1A and 1B. The devices of the present invention are preferably used in a method of providing transabdominal access for cardiac surgery. In this method, upper and lower spreader portions 4 and 6 are inserted through an incision 38 such as described above, or otherwise produced in the upper abdominal area 40, for access to the thoracic cavity, while not penetrating the abdominal cavity. The diaphragm of a patient separates the thoracic cavity from the abdominal cavity. The heart and lungs are located in the thoracic cavity and are thus located above or superior to the diaphragm. The abdominal cavity is located below or inferior to the diaphragm. The diaphragm can be described as a muscular sheet that is domed upwardly, and which is attached frontally to and along the sides to the lower rib cage margins, and obliquely near the spine in the rear of the patient. Upon the initial incision, after penetrating the skin, the surgeon carefully works along the front lower margin of the ribs, being careful not to open the abdominal cavity/peroneal sac, but gaining access to the thoracic cavity. The frontal attachment of the diaphragm is dissected away from the ribs to access the thoracic cavity, which will present as a very narrow wedge pointing toward the surgeon as he/she looks toward the patient's head from a low angle.

In using the embodiment shown in FIGS. 1A and 1B, the shell 34 is pushed into the body opening to create a working space around the heart 42. In other embodiments of the apparatus of the invention, the substernal space is created differently. However, all embodiments produce a substernal space by lifting the lower ribcage margin 12 while the upper abdomen is depressed around and at the point of incision therein, moving tissue below and adjacent to the heart away, to provide access to the heart.

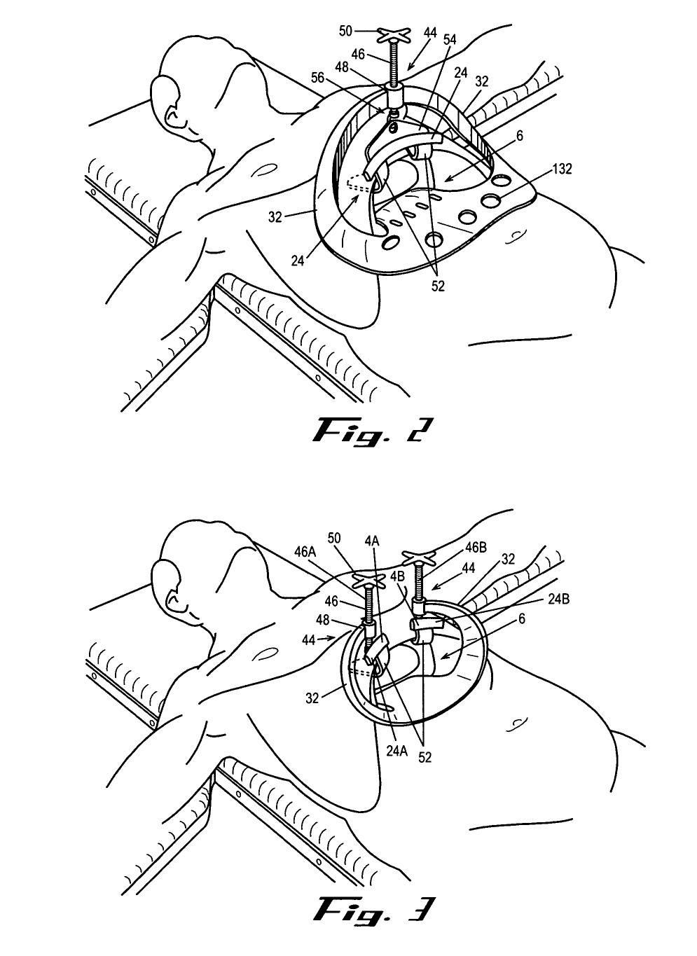

Turning to FIGS. 2 and 3, embodiments of the invention are shown which comprise an upper spreader portion 4, side body portions 32 and lower spreader portions 6, similar to the embodiment shown in FIGS. 1A and 1B. The devices shown in FIGS. 2 and 3 further comprise retraction mechanisms 44 for lifting and retracting the patient's rib cage. The upper spreader portion 4 of the apparatus shown in FIG. 2 is actuated by a single screw 46 within a threaded lug 48 turned by a handle 50. Twisting the screw 46 causes upper spreader portion 4 to lift the rib cage at the incision. The upper spreader portion 4 of the devices shown in FIGS. 2 and 3 utilize a plurality of hooks 52, each hook 52 including a lower portion 10 to lift patient anatomy and an upper portion 22 angled relative to the lower portion 10 of hook 52 to limit advancement of the upper spreader portion 4. The upper portion 22 of hook 52 provides a structural connection to associated hardware of the device.

In the embodiment of the invention illustrated in FIG. 2, the hooks 52 utilized in the upper spreader portion 4 are connected to a common mounting platform 54 optimally connected to screw 46 with a swivel and hook 56. In the embodiment of the invention shown in FIG. 3, the upper spreader portion 4 forms two discrete upper spreader portions 4A and 4B, each upper spreader portion 4A, 4B comprising hook members 52. Each spreader portion piece 4A and 4B is actuated with a separate screw 46A and 46B. A measure of independent adjustability of upper spreader portion 4A and 4B may be advantageous in handling different compliance in a patient's or subject's anatomy. Such an approach also facilitates differential opening of the cavity formed, e.g., allowing different levels of lifting or asymmetric lifting of associated ribs.

Optional hardware hook mounting rails 24 may be included in upper spreader portion 4 in the embodiments of the invention shown in FIGS. 2 and 3. In FIG. 3, two smaller rails 24A and 24B, or what may be regarded as a multi-part rail is included in the upper spreader portions 4A and 4B, respectively. Even where not shown, most embodiments of the invention may include hardware mounting rails conveniently located.

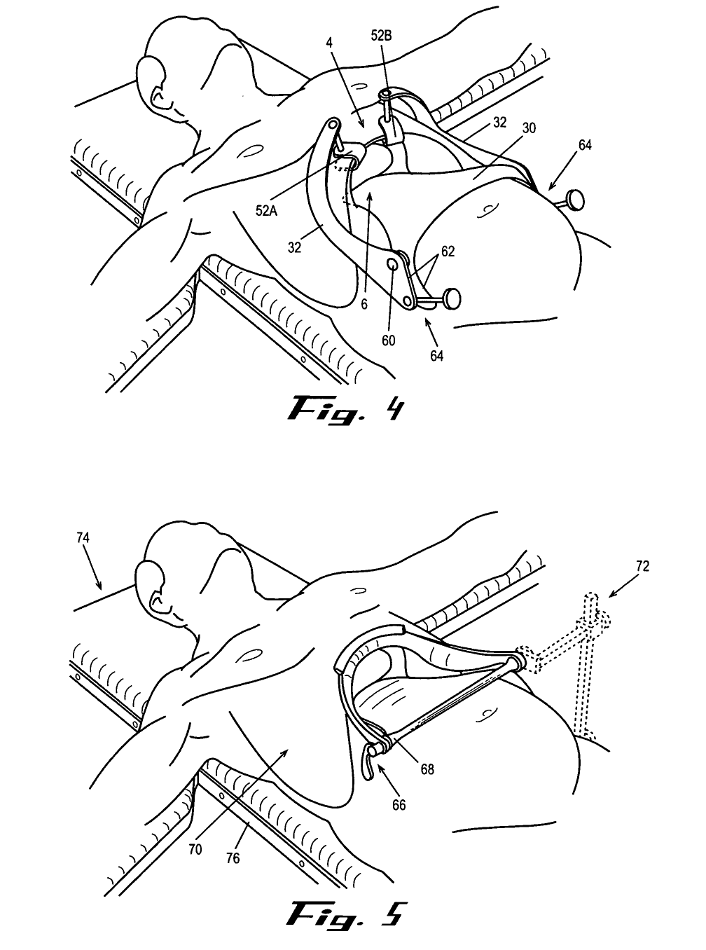

FIG. 4 shows another embodiment of the apparatus of the invention. Like the embodiment in FIG. 3, the upper spreader portion 4 has discrete sections and includes independently adjustable hooks 52A and 52B. The device shown in FIG. 4 comprises side body members 32 which move relative to the lower spreader portion 6 of the device to create a substernal access cavity. The lower spreader portion 6 of the device includes a depressor or body member 30. The side body members 32 pivot relative to the lower spreader portion 6. Extensions 62 beyond pivots 60 act as lever arms actuated by screw mechanisms 64 on either side body members 32 of the device to connect the side body members 32 to body member 30. As with other screw-type mechanisms described herein, such hardware may be replaced by ratchet-type mechanisms in instances where forced advancement or retraction is desired. Clamp-style or pin and clevis-type arrangements may be used instead to simply hold elements in place at a set separation.

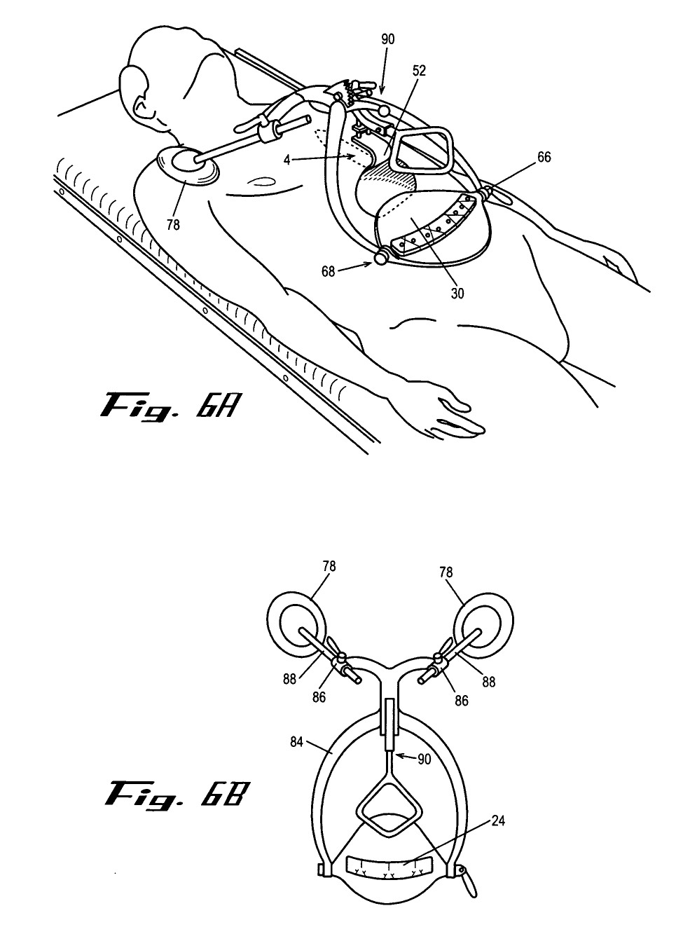

FIG. 5 shows a device much like the embodiment of the invention shown in FIG. 1, except that the upper 4 and lower 6 separator portions are adjustable relative to each other by way of a pivoting arrangement. In the embodiment shown in FIG. 5, a skewer-type clamping mechanism 66 secures the relative position of the upper 4 and lower 6 separation portions by widening or narrowing the separation between the side body elements 32 through rotation about a common pivoting interface 68. The embodiment in FIG. 1 relies on the advancement of the device into the thoracic cavity to form the desired substernal access space and the embodiments shown in FIGS. 2-4 utilize active retraction mechanisms. With the embodiment illustrated in FIG. 5, the thoracic cavity 70 is manually urged open to lift and depress the patient's anatomy to a preferable position and then locked in place by skewer-type clamping mechanism 66. Such an approach provides adjustability without particularly complex hardware.

As shown in FIG. 5, further adjustability is offered using an optional stand or mount 72. Optionally the mount is locked to a surgical table 74 along a rail 76. Well-known types of lockdown hardware may be employed for this purpose. By locking the transabdominal access device to a fixed point, reaction forces are provided allowing for asymmetric loading of the upper separator portion 4 to the lower separator portion 6. Anchoring the transabdominal device externally may also help stabilize the surgical field. It should also be noted that achieving system adjustability and stability may be accomplished in other ways than shown in FIG. 5.

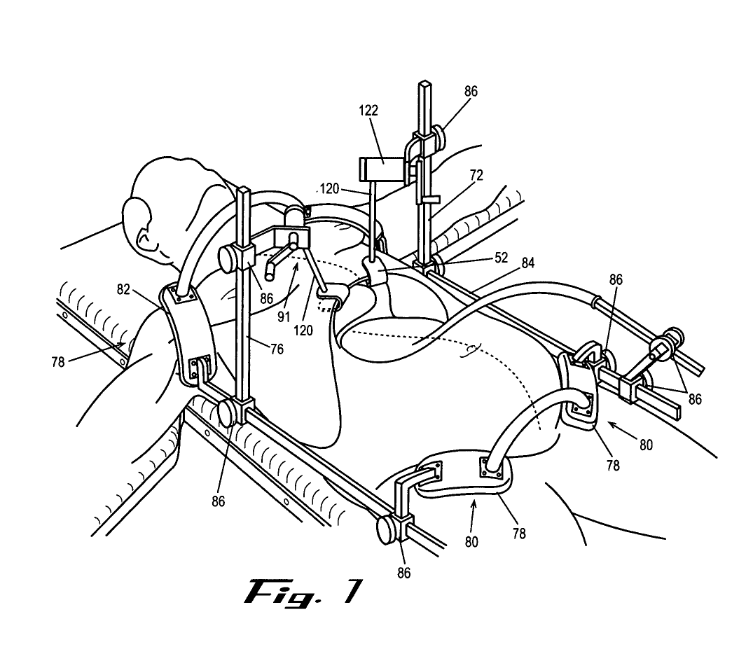

FIGS. 6A and 6B show views of another embodiment of the apparatus of the invention comprising contact pads 78 which are configured to stabilized the device against a patient's shoulder regions 80 as well as the abdomen. The embodiment shown in FIG. 7 is braced similarly against the body o f the patient, except that the device is braced at the shoulders and pelvic area or upper thigh of the patient.

These approaches to stabilizing the device also facilitate lifting of the ribs with significant force. In each of the embodiments of the invention shown in FIGS. 6 and 7, the underside of contact pads 78 provide reaction force against the patient's body while the ribs and sternal area are lifted. In the embodiments shown in FIGS. 6A and 6B depressor 30 alone works in conjunction with the pads 78 to provide the reaction force. In the embodiment shown in FIG. 7, lower contact pads 78 positioned on the upper thigh 82 provide additional system flexibility and stability. As shown in FIG. 7, the position of various elements of the device may be adjusted along frame 84 and mount 72 by clamps 86.

In the embodiment of the device in FIGS. 6A and 6B, the size of the frame or side portions 84 of the device are adjusted by clamped sliders 88. The lower spreader portion 6 may be setup for pivotal adjustment as well. As with the embodiment shown in FIG. 5, adjustment of the pivotal orientation of the depressor surface 31 may be achieved with a pivoting interface 68 locked down by a clamping mechanism 66.

The lower spreader portion 6 shown in FIG. 6 includes an optional built in stabilizer rail 24 with suture holder. This specific type of rail for optionally mounting hardware and restraining sutures is more fully described in U.S. Pat. No. 6,283,912 to Hu et al. Of course, such a feature may be used in other locations in this embodiment (e.g., mounted on the upper separator portion) or in other embodiments.

In the embodiment of the invention in FIGS. 6A and 6B, the upper spreader portion 4 comprises a ratchet mechanism with a removable handle 90 for generating a desired force to lift the sternum and maintain the position of the sternum, together with surrounding tissue, in a locked position. The handle 90 is preferably removable to avoid interfering with the surgical field once the position of the upper spreader portion 4 is set. Instead of a ratcheting mechanism, a screw-type mechanism, piston arrangement or other forcing mechanism such as a hoist or a winch like those shown in FIGS. 7 and 10-16 may also be used.

The upper spreader portion 4 shown in FIG. 7 is separated into two sections with each section comprising a hook 52 configured to a winch-type mechanism 91 for lifting the sternum. The winch type mechanism 91 is positioned along mounts 72 by clamps 86. The winch type mechanism shown in FIG. 7 is described in further detail below.

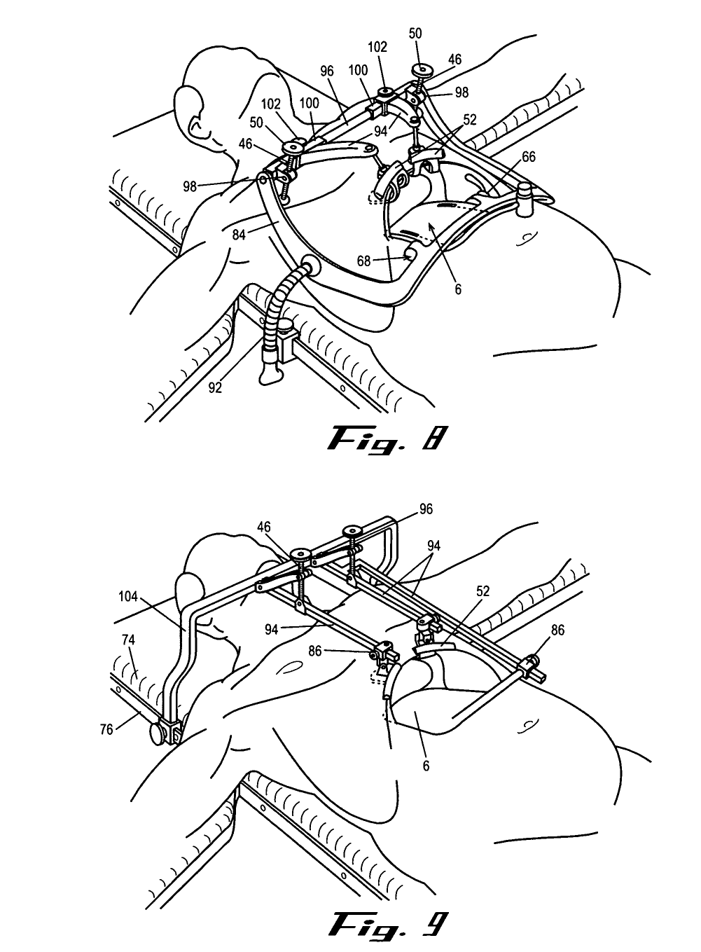

Like the embodiment in FIGS. 6A and 6B, the embodiment of the invention shown in FIG. 8 includes a settable, variable-tilt lower spreader portion 6. However, instead of making contact with the body of the patent to stabilize itself against movement and for mechanically retracting the lower ribcage margin, it is stabilized along its frame 84, preferably on both sides of the patient. A locking multi-link device 92 such as described in co-pending, commonly assigned application Ser. No. 09/769,964, for example, or the like, may be used. Otherwise, standard bracketing or framing like that seen in other Figs. or as is common in the art may be employed.

The embodiment of the invention in FIG. 8 utilizes screw-type adjustment features to control lifting or retraction of the upper spreader portion 4 of the system. As in other embodiments, the upper spreader 4 shown in FIG. 8 comprises discrete hooked pieces 52. They are each independently driven by separate screws 46 operated by knobs 50. Each hook piece 52 is tethered to a boom or actuator arm 94. These arms extend from a cross member 96 about which they can pivot. The vertical position of each actuator arm 94 is set by screw 46 via rotatably mounted lugs 98 offset from the actuator arm pivot axis along cross member 96.

The location of the arms 94 relative to cross-member 96 may be varied along each slider 100 and set via clamp 102, possibly employing a set screw or some other securing mechanism. The ability to vary the spacing of the actuator arms 94, and thus the retractor pieces 52 is useful to account for different sized patients.

While the embodiment of the invention shown in FIG. 9 does not show crosswise or side-to-side adjustment features, they may b e incorporated in such an apparatus as well as many others described herein. The embodiment shown in FIG. 9 does provide features for flexibility which are applicable to other systems/devices herein as well. The embodiment shown in FIG. 9 is configured to allow the in-and-out or forward-and-back distance between the lower spreader portion 6 and hooks 52 of the upper separator sections to be varied. Clamp members 86 allow the hooks 52 to be independently adjusted along each actuator arm 94. Adjustability in hook 52 spacing may be used to account for variation in patient size. The ease of adjusting hooks 52 and the lower spreader portion 6 by clamp members 86, also allows for easy removal of the components of the device. This may be a benefit in order to handle sterilization issues, replace broken parts or simply change out certain components for components of varying size, depending on the type of surgery and/or the size of the patient.

As shown in FIG. 9, each arm 94 is set in place and/or driven to a desired height or depth relative to the patient by way of screws 46. The force generated thereby, or with another driver mechanism, is handled by frame 104. It is shown attached to surgical table 74 along its rails 76. As in other setups according to the present invention, the location of the surgical table attachment may be altered to provide additional flexibility.

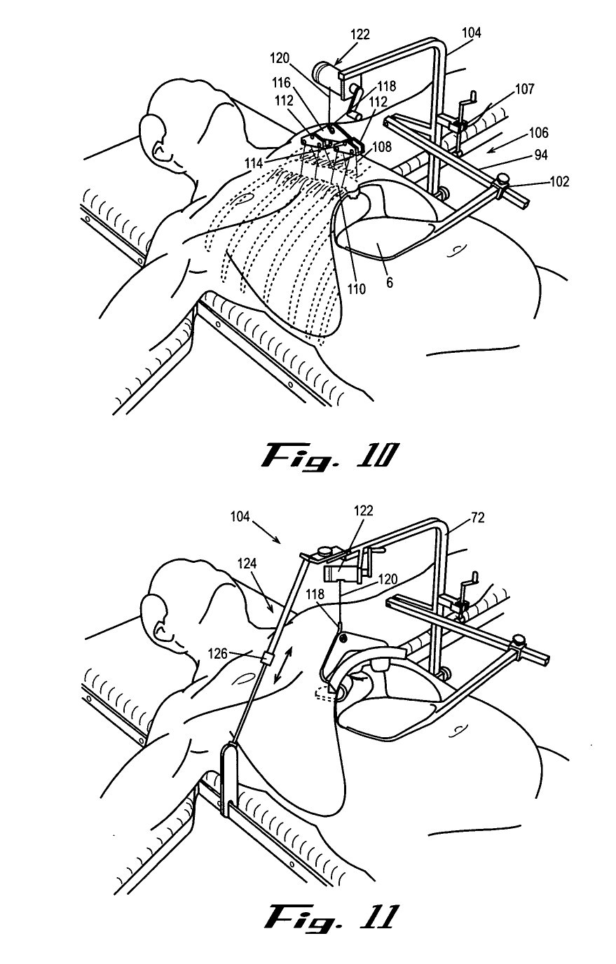

FIG. 10 shows another embodiment of the system according to the present invention that is advantageously table-mounted. This embodiment utilizes a lower separator armature 106 similar to an actuator arm 94 shown in FIG. 9. A screw drive 107 is used to locate the depressor surface 30 as desired. However, the embodiment shown in FIG. 10 utilizes a different sort of interface for the upper separator portion 4 which eliminates certain hardware thus providing more room at the incision site as well as for activities such as LIMA harvesting.

Rather than relying on a flat lift surface such as a hook, the sternal area is lifted using cables, such as sternal wires like those use to close a cut sternum or tension strands 108 running through the patient's chest in the embodiment shown in FIG. 10. Strands 108 preferably comprise wires or heavy suture material threaded under the sternum through adjacent intercostal spaces 110 forming a sling as the upper spreader portion of the system. Strands 108 are shown received by shackle members or sling fittings 112. Preferably, pins 114 rotatably connect the fittings 112 to an optional plate 116. The plate 116 is used to bridge fitting 114 and optional hook 118. Plate 116, in conjunction with fittings 112 ensure the automatic, even tensioning of strands 108. The hook 118 is connected to a tether 120 actuated by a hoist or winch 122.

A manually driven winch is shown in FIGS. 7, 10-12. However, a motor driven unit may be substituted to lift the sternal area. Further, the plate 116 and/or hook 118 shown in FIG. 10 may be omitted in favor of a direct connection of the tether to a single sling fitting. The setup shown in FIG. 10 is thought to be more advantageous, from the perspective of ease of use, especially by the manner in which the independently mounted sling fittings 112 assume an equilibrium position upon tension being applied, resulting in roughly equal pressure applied to the strands (of a similar diameter) in order to reduce trauma to the tissue.

FIG. 11 shows another embodiment of the invention utilizing a hoist mechanism for the upper spreader portion 4 and a lower spreader portion 6 arrangement similar to the embodiment shown in FIG. 10. However, the upper spreader portion 4 is a dual-hook lifting member instead of the sling arrangement shown in FIG. 10. Furthermore, the frame 104 of the embodiment shown in FIG. 11 further includes an additional brace member 124 configured to the hoist mechanism. The brace member 124 is adjustable in an axial direction by a screw-type wedge mechanism 126 or the like. The brace 124 is preferably attached to the table and the hoist stand or mount 72, together forming a complete frame 104.

The embodiment of the invention shown in FIG. 12 utilizes a frame 104 which includes over-the-shoulder braces 128 attached to table rail 76. This configuration allows mount arms 72 to be securely mounted further back from the transabdominal incision 38. Such a location for the mount arms 72 and concomitant forward-mounting of hoist mechanism 122 relative to the patient, allows the upper lifter portion(s) of the device to draw the lower ribcage margin up and away for more efficient exposure of the heart. In addition, the embodiment shown in FIG. 12 includes an optional feature, a ratchet mechanism 130 for driving and/or maintaining the depressor member 30 in place.

FIG. 13 shows various instruments as may be desired in performing cardiac surgery transabdominally with the present invention. A lower spreader portion 6 is shown that includes instrument mounting features 132 for such tools. Additional mounting features 132 may be provided or positioned in alternative locations of the device. The device shown in FIG. 13, further comprises a manipulator 134 that is captured by one such mount 132, and is configured to engage the heart, e.g., at the apex region of the heart, such as by vacuum and/or other attachment means, to allow an operator to position the heart into a desired orientation for performing a procedure. Examples of manipulators that may be employed are described in U.S. Pat. Nos. 6,338,712; 6,390,976; and 6,506,149; as well as co-pending, commonly assigned application Ser. No. 10/615,007 filed Jul. 8, 2003 and titled “Organ Manipulator Apparatus”.

A lighted scope 136 is shown just outside of a mount. A pair of stabilizers 138 such as described in U.S. Pat. No. 6,036,641, for example are also shown. The upper stabilizer reaches the heart through a port 140 made in the chest. The second reaches the heart through the transabdominal cavity 36. It is affixed to a rail 24 provided in connection with hook member 52 byway of a rail lock 142 such as described in co-pending, commonly assigned application Ser. No. 09/958,263 filed Mar. 6, 2002 and titled “Surgical Instruments for Accessing and Stabilizing a Localized Portion of a Beating Heart”, for example. Tethers 120 are shown retracting the upper spreader portion of the system formed by hooks 52 to lift the patent's sternal area.

A point-and-shoot “gun” 144 for producing a proximal anastomosis between a graft and patient aorta 146 is also shown. Such a device and associated anastomosis hardware are described in application Ser. No. (application Ser. No. not yet assigned, Attorney's Docket No. GUID-037) titled “Anastomosis Device, Tools and Methods of Using” filed Dec. 24, 2003. application Ser. No. (application Ser. No. not yet assigned, Attorney's Docket No. GUID-037) is incorporated herein, in its entirety, by reference thereto. Access to the heart is provided by a second access port 140. Preferably, the ports are produced through intercostal spaces. Actually, producing the proximal anastomosis may be accomplished in any number of ways such as those noted in the Background section above, by robotic surgery methods or as otherwise apparent to those with skill in the art.

In carrying out a CABG procedure, the distal anasotmosis may be made with a coronary artery 148 held by a stabilizer 138 as shown. Due to the size of the substernal cavity created, in many cases, it is feasible to use typical surgical techniques to produce the anasotmosis. However, it may be preferred to use connector systems or approaches utilized with proximal anastomosis, or specifically designed for performing distal anastomoses.

The techniques and instruments described in association with FIG. 13 are equally applicable in connection with the embodiments of the invention shown in FIGS. 14-20. However, each of these embodiments shows an aspect not present in the embodiments of the invention described thus far. Namely, the later embodiments include sideways rib compression features that assist in substernal cavity formation. By pressing inwardly on the ribcage while lifting the sternal area, greater extension of the space can be achieved. This allows more vertical working room for a surgeon or a surgical team, as well as surgical tools (.e.g., those described so far or robotic componentry, or other tools typically used for such procedures).

The embodiments of the invention shown in FIGS. 14 and 15 include many of the features described thus far, and further include side compression plates or pads 150 to side portions of the device. The compression plates 150A and 150B are preferably curved to conform to the shape of the patient's ribcage. The compression plates 150A and 150B include compression surfaces 152A and 152B which contact the sides 154 of a patient's thorax 156 and may be padded in certain embodiments. The compression plates 150A and 150B each also include an outer compression plate surface 153A and 153B, respectively. Outer compression plate surfaces 153A and 153B are mounted to respective horizontal rails 158 by advancement mechanisms 160 which are configured to adjust the amount of compression the compression surfaces 152A and 152B apply to the patient's sides.

In the embodiments shown in FIGS. 14 and 15, compressing the compression pads 150A and 150B and thus compressing the patients thorax is accomplished by advancement mechanisms 160 which includes screws 46 that are operatively connected to the outer compression plate surfaces 153A and 153B. The advancement mechanism 160 also includes lugs 48 that are adjustable along the horizontal sliders 158 to optimize placement of compression plates 150A and 150B relative to the lifting or upper portion(s) 4 of the apparatus. Clamps 86 may be used to secure the lugs 48 to the horizontal sliders 158 to insure the horizontal location of the compression plates 150A and 150B. The screws 46 shown in FIG. 14 are actuated by crank-type handles 50 while those in illustrated in FIG. 15 are actuated by knob-type handles 50. Advancement mechanism 160 further may include a locking element 162 to fixedly set the position of pad 150.

Vertical adjustment of pads 150A and 150B is achieved by sliding lugs 86 along the side portions or sections of the frame 104 of the lifting portion of the apparatus. Clamps may be used to the secure lugs 86 and insure the vertical location of the compression plates 150A and 150B. The embodiment shown in FIG. 15, shows the side portions of the lifting portion of the apparatus coupled to table mount rail 76 for further stability of the device.

The embodiment of the invention in FIG. 16 utilizes rib-compression features 150 that are configured to coordinate side or rib compression with sternal lifting. Coordination of side compression and sternal lifting is accomplished by a linkage assembly 164. A pair of rocker arms 166A and 166B are pivotally attached to frame 103 for further stability of the linkage assembly 164. The pivoting members 167 are similar to those described in other embodiments of the in the invention and may be bearings such as plastic bearings, DU® bearings, cartridge bearings or the like, and may be used in conjunction with any sort of a pin or shoulder bolt. In the event pins are used, they may be secured in place via snap rings or otherwise. The device shown in FIG. 16 includes compression members 150A and 150B mounted near a first end of each rocker arm 166A and 166B.

Optionally, a screw-type adjustment device 165 is configured to the compression member 150 to allow for adjustment of the location of the compression surface 152 relative to the rocker arm 166 (e.g., see FIG. 16). The embodiment shown in FIG. 16 additionally includes, a turnbuckle 168 attached to the inner end or second end of each rocker arm 164 at the opposite sides to a lift member assembly 170 for adjusting the position of the rocker arm(s) 166 relative to the lift member assembly 170. A simple linkage system may be substituted for either one or both turnbuckles 168. The vertical height of the placement of the compression pad 150 may be varied by sliding clamps 86 along a crossbar 96 of the frame 103.

The lift member assembly 170 preferably comprises a screw 46 rotatably attached to a runner 172 with a groove 174. The groove 174 is located about a fixed lug 48 through which the screw 46 is threaded. By turning knob 50 the lift member assembly 170 is raised, retracting the upper spreader portion 4 and thus retracting/lifting the sternal area. Simultaneously, compression pads or plates 150 constrict or compress the ribcage promoting lifting of the sternum as the ribs are flexed to provide a transabdominal cavity.

The lower separator/depressor portion or member 6 shown in FIG. 16 is also positioned relative to the patient's body in a unique way. Here, an actuator arm 94 is articulated by a screw-driven four-bar linkage 175. The linkage 175 is mounted to the cross bar 96 of frame 103 by a clamp 84, allowing for side-to-side adjustment of the lower spreader portion 6. By providing the four-bar linkage 175 to actuate the arm 94, vertical or up-and-down motion of the lower spreader portion 6 can be controlled to limit rotational movement of the depressor surface 31. Instead, rotational or pitch adjustment is provided by way of a pivoting interface 68. The position of the pivoting interface 68 is set, as is convenient for surgery, by a clamp 86.

The lower separator section 6 of the embodiment of the invention shown in FIG. 16 includes at least one or more features of note. Particularly, the depressor 30 of the lower separator section 6 shown in FIG. 16 has a non-planar shape. The embodiment shown in FIG. 16 also includes upturned wing portions 176 which help open the sides of the subabdominal cavity for better surgical access. Further, the depressor 30 may optionally include a down-turned or hooked nose portion (not shown in FIG. 16). This concave-down portion provides additional clearance within the substernal space by depressing tissue toward the patient pelvic area. Also, both FIGS. 15 and 16 show recessed through slots 134 or other suture retaining features. Suture retaining features 134 may be used for one method of lifting/exposing the heart, particularly the ventral surface of the heart. In such a procedure, deep needle bites are taken on the top rear surface of the diaphragm. The needles, which are connected to strong sutures are then drawn out of the diaphragm and downward, across the depressor 30/separator section 6, tensioned to effect the desired exposure, and then the sutures are retained in retaining features 134. Retaining features 134 may optionally include suture locks to maintain the desired tension on the sutures, or the sutures may be anchored to any convenient features below the slots 134. An example of suture locks that may be employed is disclosed in U.S. Pat. No. 6,283,912, which is incorporated herein, in its entirety, by reference thereto. Although illustrated with regard to FIGS. 15 and 16, recessed through slots 134 or other suture retaining features, including suture locks, may be provided with any of the examples described herein.

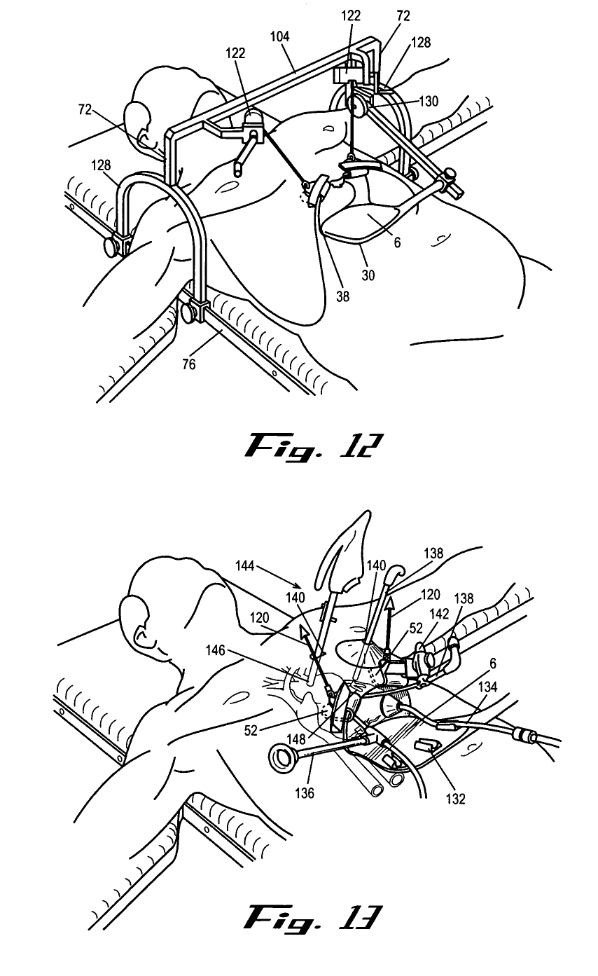

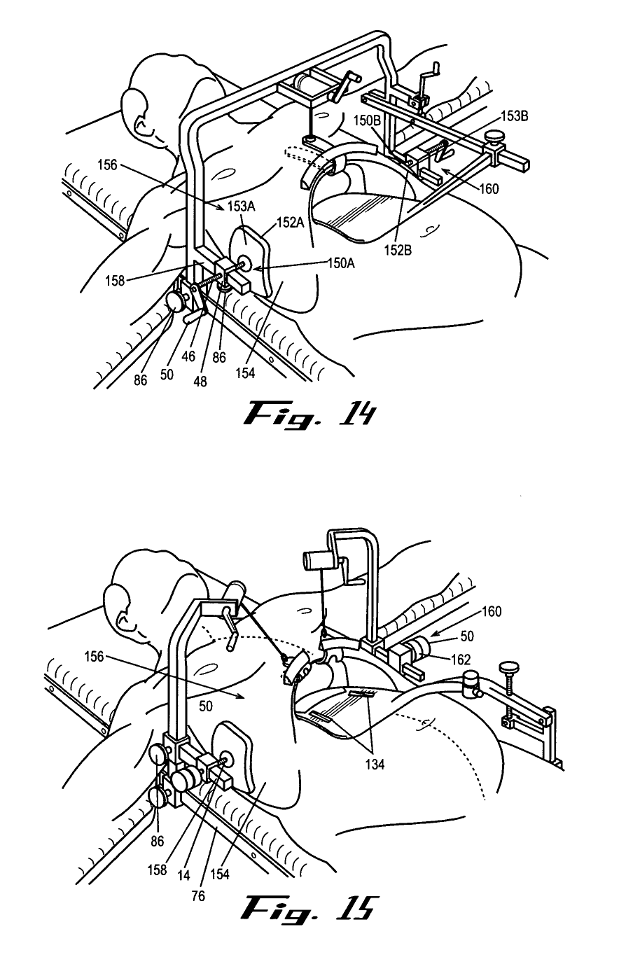

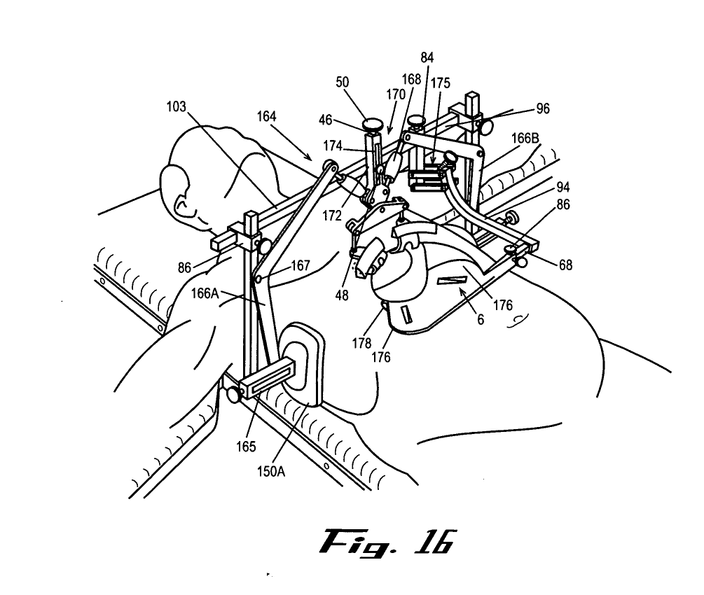

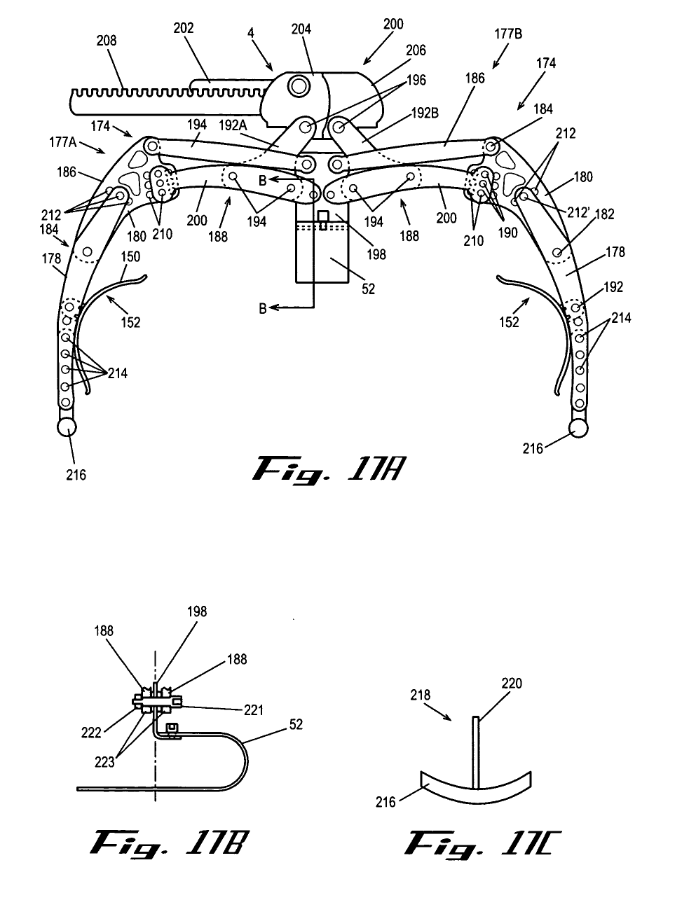

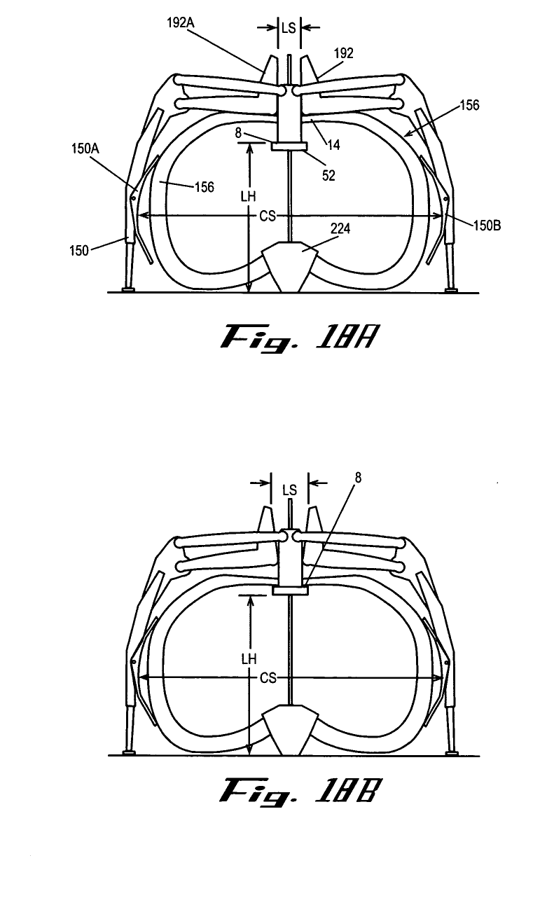

FIGS. 17A and 17B, show another embodiment of the upper spreader portion 4 of the invention. In this embodiment, the upper spreader portion 4 and compression pads 150 of the system are coupled by a symmetrical flexible linkage setup which when employed, compresses the ribs sideways and lifts the sternal area in a coordinated fashion. The flexible linkage setup comprises two linkage assemblies 177A and 177B which are arranged to be bilaterally symmetrical or mirror images of each other.

The linkage assembly 177A and 177B comprises a first linkage member or arm 178 and a second linkage member 180 pivotally coupled thereto by a pin 182 to form a linkage as shown in FIG. 17A. The second linkage member 180 has a first end coupled to first linkage member 178 and a second end which is pivotally coupled thereto by a pin 184 to a third linkage member 186. The second linkage member 180 also is pivotally coupled to a forth linkage member 188 by a plurality of pins 190 at the second end of the second linkage member 180. The third linkage member 186 is operatively positioned adjacent or above to the fourth linkage member 188 to aid in rib compression and sternal lifting.

The apparatus further comprises movable lever arms 192A and 192B which are fixedly coupled to respective forth linkage members 188 by a plurality of pins 194. The shape of the movable lever arms 192A and 192B is essentially triangular, such that two corners of the triangle are coupled to forth linkage members 188 and the third corner or upper most portion of the movable lever arms 192 are pivotally coupled to the upper spreader portion 4 of the device by pins 196 thereto. By moving the lever arms 192A and 192B apart from each other, linkage assemblies 177A and 177B are actuated, which in turn decreases the distance between the first linkage members 178 while simultaneously elevating the upper portion 4 of the invention.

The upper spreader portion 4 of the device includes a main body member 198 and hook 52 operatively configured to lift the sternum region of the patient. The main body member 198 is pivotally coupled to both linkage assemblies 177A and 177B to allow for even rib compression from compression pads 150A and 150B and simultaneous lifting by hook 52. While separate bolt-together pieces are shown in FIG. 17B for coupling the main body member 198 and the hook 52, an integrated unit may be used. However, separable portions of the main body member 198 and the hook 52 may be preferred considering sterilization needs and interchangeability options offered thereby.

Each of the third linkage members 186 is shown in FIG. 17A to be attached to both a second linkage member 180 and pivotally coupled to the main body member 198 of upper spreader portion 4 without provision for varying its attachment location to main body member 198. However, an adjustable arrangement may be provided in connecting the third linkage members 186 to the main body member 198. Each forth linkage member 188 is also pivotally coupled to the main body member 198 in a fixed location to allow coordinated movement of the third and forth linkage members (186 and 188, respectively) when the main body member 198 of is lifted or lowered.

The upper spreader portion 4 further comprises a rack-type mechanism 200 configured to main body 198 and linkage assemblies 177A and 177B for adjusting rib compression and sternal lifting. The rack-type mechanism 200 includes a latch or lever arm 202, a first side 204 and a second side 206, the first side 204 being movable over rack 208 relative to the second side 206 of the rack-type mechanism 200.

The adjustment element 202 may be a lever arm or cam system which is configured to the first and second side (204 and 206, respectively) of the rack-type mechanism 200 to lock the first and second sides of the rack-type mechanism into the desired position along rack 208. Further details to the operation of a driving device of this type are described, for example, in U.S. Pat. No. 6,231,506, which is incorporated herein, in its entirety, by reference thereto. Various types of driving devices such as screw drives, hydraulic drives and the like may be substituted for the driving device 200 shown in FIGS. 17A and 17B.

The movable lever arms 192A and 192B of the embodiment shown in FIG. 17A are pivotally coupled to the first side 204 and the second side 206 of rack-type mechanism 200, respectively, by pins or bolts 196. The lever arms 192A and 192B are preferably driven at equal elevation at pivot points and pins 196. Adjustment of the relationship between the lifting hook 52 and compression members 150A and 150B may be altered in various ways.

Since rotation is required between each of the links described in the embodiment shown in FIG. 17A, connection of the linkage members is achieved via a pin, shoulder bolt or like element. To allow angular adjustment of a link formed by linkage members, coupled linkage members may comprise a series of leverage adjustment positions such as the leverage adjustment positions 210 of the second and forth linkage members 180 and 188 shown in FIG. 17A. Preferably only one set of the leverage adjustment positions 210 is chosen to link or couple the second 180 and forth 188 linkage members together. The multiple leverage adjustment positions 210 allow the operator to chose an appropriate amount of compression and stance of first linkage member 178 and the attached compression pad 150.

In certain embodiments, the stance and vertical position of first linkage member 178 may further be aided by including a plurality of adjustment positions 212 in the first linkage member 178 and the second linkage member 180, where the first and second linkage member are coupled there through a specific or chosen adjustment position 212′ by a bolt or pin which fixedly positions the first and second linkage members about pivot pin 182. Leverage adjustments 210 and 212 provide a variety of positions for the side portions of the device shown in FIGS. 17A-C.

Each first linkage member 178 further comprises a plurality of compression pad mounting holes or openings 214 where at least one rib compressor pad 150A and 150B can be operatively mounted to the first linkage element or arm 178. The purpose of the link 182 formed by first linkage member 178 and second linkage member 180 is to allow adjustment of compressor pad 150A and 150B positioning with respect to the patient by altering which compression pad mounting hole 214 is chosen to orient the lower portion of the compression pad 150 about pivot 214. Of course, other adjustment approaches are possible to set compressor pad 150 spacing. However, the approach shown in FIG. 17A is both convenient and allows for quick changes by simply altering the tie-in point of a couple of pivot pins or shoulder bolts 214. Further, holes 214 allow for a length adjustment of foot portions 216 which telescope with respect to members 178.

Optional feet 216 for stability of the upper spreader portion 4 and compression pads 150 of the system may be provided as shown in FIGS. 17A and 17C. The feet 216 may be affixed to a stationary item such as the surgical table or its rails in order to provide a reaction force to lifting by the hook 52. Alternately, they may simply rest against a surface, such as that of the surgical table to set the height of the device via various mounting locations 218 or angle relative to the patient's body. Likewise, the height of each compressor pad 150 relative to the device's feet 216 may be adjusted by rotably affixing it to any one of a number of mounting locations 218.

The feet 216 may be blunt or curved such as shown in FIG. 17C to allow rocking of the device to set the device to desired angles during use. On the other hand, the feet 216 may be straight and set or clamped at an angle relative to its leg 220 to accomplish setting the device at the desired angle.

While the device may only include one layer or set of certain link pieces, it is also contemplated that two or more layers may be provided for increased rigidity or stability or to permit symmetric loading of bearing surfaces. Preferably, two of pieces 178, 186 and 188 are provided on each side of the device shown in FIG. 17A. Put another way, these link members are preferably “paired.”

Pairing also facilitates the connection of features as shown in FIG. 17B. Here each third link member 186 (not shown in FIG. 17B) and fourth link member 188 sandwiches movable lever arm 192 as well as second link member 180. A shoulder bolt 221 captured by a nut 222 secures the items in place. Thrust bearings 223 are provided to avoid surface wear between the components. Though not shown, bearings may also be provided around the body of the connector.

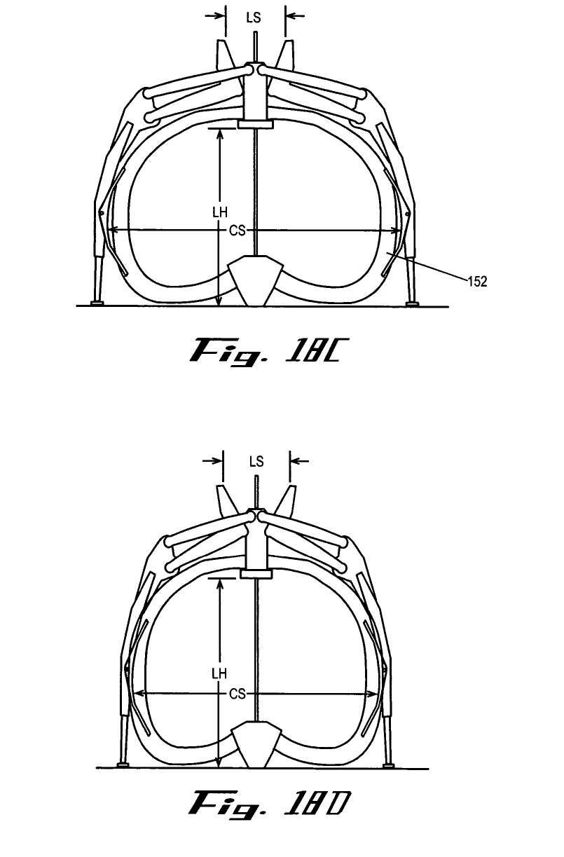

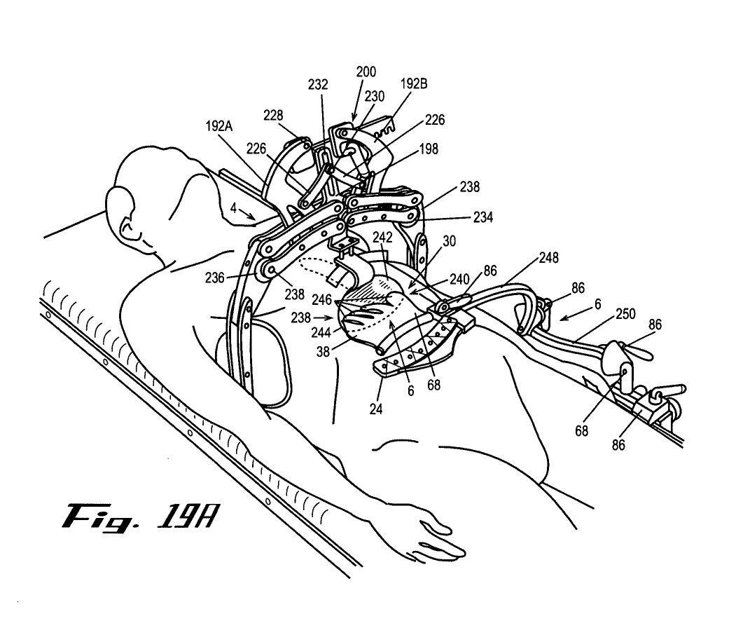

The operation of the device shown in FIGS. 17A-17C is illustrated in FIGS. 18A-18D. A model of the thorax 156 shown in cross section is used to demonstrate the action of the device. In FIG. 18A, lift surface 8 or hook 52 is shown below the sternum area 14, and compressors 150A and 150B are also shown separated some distance from the sides of the thorax 156. FIG. 18A also shows the vertebra 224 of the model. No drive mechanism is shown between lever arms 192A and 192B in FIGS. 18A-18D so as not the obscure the relationship of the lever arms 192A and 192B through the stages of operation of the device. In FIGS. 18A-D, the separation between lever drive points 196 is shown as LS, the separation between the side compression surfaces 152 is labeled CS and the height of the lifting surface 8 from a horizontal such as a table 74 surface is labeled LH for clarity.

FIG. 18B shows first contact of the compress surface 152 and lift surface 8 with the modeled rib cage structure. Preferably, the contact surfaces are coordinated to simultaneously contact the patient, however, other situations are contemplated.

FIG. 18C shows significant compression of the model thorax 156. The squat, oval shape increases in the direction of a minor axis (i.e., LH increases) and decreases in the direction of a major axis (i.e., CS decreases). This occurs as the result of increasing the size of LS by such drive mechanism as may be provided.

FIG. 18D shows full extension of LH, compression of CS and separation of LS in order to form as tall of a substernal space as feasible. In reality, a better fit of the compression surfaces with the side of the ribs will be observed. This may be accomplished through the use of flexible curved members 150 that flatten in response to the ribs loosing curvature or flattening upon compression as observed in comparing FIGS. 18A-18D.

Utilizing both side compression and lifting (whether with the embodiments of the invention pictured or otherwise accomplished), it is possible to lift the sternum of an average size adult patient upwardly by about one to three inches. Rib compression facilitates such lifting and makes it possible to increase the amount of such lifting. The amount of rib compression used to assist such lifting is typically between about one and four inches (total CS) Still, in some situations, such as cylindrical or “barrel-chested” rib cages, less sideways compression to the thorax may be desired, and sternum or ribcage margin lifting alone may be employed to lift the sternum. Because the greatest cross-sectional area of a closed from or shape is a circle, dorso-ventrally flattening of a cylindrical-shaped rib cage (sideways rib compression) may accomplish the opposite effect of what is desired, i.e., instead of the organs falling away and leaving working spaced for the surgeon, the internal organs may rise and fill all the created space made when the thoracic cavity is opened and air first enters the opening, partially collapsing the lungs. In such cases, with sternum or ribcage margin lifting alone, the area can be displaced upward between about one and three inches in an average “barrel-chested” patient. However accomplished, more rib cage displacement may be realized in larger chest size individuals since lower stress states are generated for a given amount of displacement. In smaller individuals, particularly children, infants and elderly patients, less displacement may be possible. Furthermore, certain situations such as a rigid ribcage; mushy, friable or brittle bones and/or sternum; or anomalous chest shapes may call for less than maximum sternal/lower rib cage extension to create a maximum size substernal space. The ratio of lift to side compression is completely customizable to meet these needs.

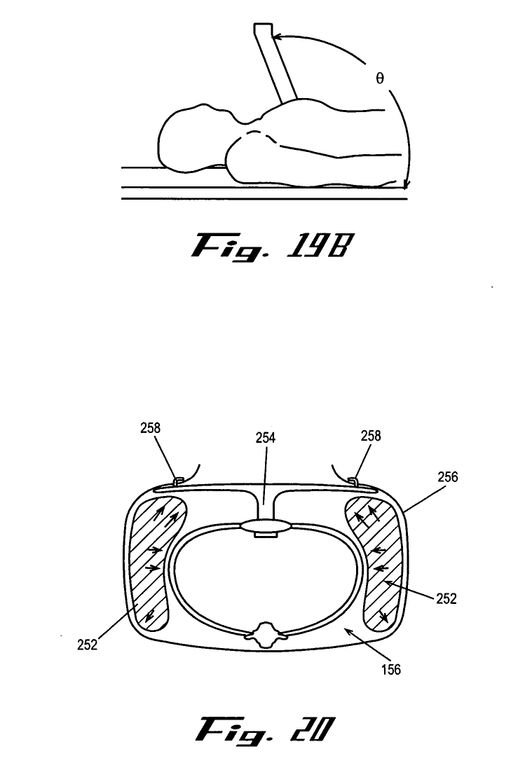

FIGS. 19A and 19B show an alternate linkage type embodiment of the invention. Separate lift and depressor units, 4 and 6, respectively, are employed in a single transabdominal access spreader system according to the invention. The lift mechanism differs most from that shown in FIG. 17A by the addition of stabilizer links 226, and an associated track 228 to keep the lever arms centered, as well as to keep the whole retractor mechanism centered, to prevent it from racking sideways when forces, e.g., such as those which might be imposed by an uneven or asymmetrical ribcage, are imposed. A pin or another sort of key 230 rides in the track groove or way 232, the track 228 is preferably formed as an extension off of the main body member 198.

To avoid interference with the track and associated links formed by the linkage members, the lever arm drive mounts 192A and 192B and the rack-type drive 200 are elevated relative to the embodiment of the invention shown in FIG. 17A. The rack drive is inverted as well in order to keep the height of the device as low as possible.

Another modification of the device shown in FIG. 19A is in the use of a cam 234 to modify linkage travel for side compressors 150/compression surface 152 and hook 52. By rotating and locking down each cam 234, the location of the offset linkage tie-in point 236 is changed. The stabilizer link setup ensures that the lever arms and compressor pads 150 are adjusted equally as desired. Still, such a cam arrangement may be employed in the embodiment of the invention in place of adjustment locations such as the adjustment locations 210 shown in FIG. 17A.

In FIG. 19A, the type of link assembly pairing described above in connection with the embodiment of the invention shown in FIG. 17A is fully illustrated. In addition, FIG. 19B demonstrates the preferred angular orientation of devices like those shown in FIGS. 17A and 17B, discussed in connection with optional foot 216 features illustrated in FIG. 17C. Regardless of whether angled foot features are provided as configured, the upper spreader or lift portion 4 of the device is preferably oriented with respect to the body of a patient prone on a table, at an angle θ ranging from about ninety-five degrees to about one hundred thirty degrees. Such an orientation results in an upward and forward displacement of the ribcage margin. Straight vertical lifting is also possible as well as mild reverse angles. However, lifting at an angle as stated above, generally provides the suitable substernal space for transabdominal access. While embodiments of the invention which do not comprise a linkage type assembly have the ability to easily retract the sternal area of the patient at a greater angle θ, the linkage mechanisms shown in FIGS. 17A-19B provide a preferred manner of lifting for providing transabdominal access.

As shown in FIG. 19A, a complete transabdominal access system is provided using a separate upper spreader portion or lifter 4 in combination with a separate lower spreader portion 6 comprising a depressor 30 whereas other embodiments according to the present invention are more integrated. The depressor 30 shown in FIG. 19A comprises a plurality of discrete fingers 238 unlike the depressor shown in FIG. 16 which has contiguous surface. Though six discrete fingers 238 are shown in FIG. 19A, the number of fingers employed may vary. The shape of the depressor 30 will be partially determined by the material utilized to produce the depressor, especially when formed of a malleable material, such as aluminum, stainless steel, metal alloy or deformable plastics, for example.

The shape and position of individual fingers 238 of the depressor 30 may be separately positioned to optimally hold down or hold back internal body tissue or organs. The inner fingers 246 of the depressor 30 have the primary function of holding down or depressing internal body tissue while the external or outer fingers 240 may serve not only to hold down internal body tissue, but may also be shaped upward at the side 244 in a cupped fashion to provide a transition space between a lower portion of the incision 38 and the sides of the incision. Further, the fingers 238 include ends 246 which are shaped downward and/or outward to restrain or depress tissue to assist in clearing substernal space 36. In the event the fingers 238 are not malleable to facilitate on-the-spot adjustment, preshaped members may be employed, and may be formed of injection-molded polymer, metals or composites selected from many different formulations that are well-known for use in the surgical fields.

Depressor 30 shown in FIG. 19A is also rotationally adjustable about a pivoting interface 68 secured by a clamp 86 to provide additional adjustability of movement and positioning of the depressor 30. The depressor 30 further comprises a first placement arm 248 and a second placement arm 250 which may be rotated about pivoting interfaces 68 and locked in place by clamps 86 to provide additional degrees of freedom of the depressor 30. The first and second placement arms 248 and 250 are operatively secured/clamped to the surgical table. It should be noted that other securing options for the depressor 30 may be utilized besides for the clamp setup shown in FIG. 19A, such as ratchet mechanisms and the like. Using ratchets or another active mechanism of that sort, facilitates compressing or depressing body tissue while locking of the device in position under greater force.

With respect to the depressor portion 30 of the system, it is also shown in FIG. 19A that the separate lower spreader portion 6 may be optionally provided separate from instrument and/or suture retention features. A rail 24, such as described above (or otherwise configured) may be mounted off of placement arm 248 to provide space for surgical instruments or suture retention features. By providing a separate area which is remote from the depressor 30, this helps clear the surgical field and provide more clearance around the incision for accessing the substernal space. Additionally, sutures may be installed to assist force application of the depressor 30. By installing sutures in the pericardium beneath the heart, for example, tension may be applied to the sutures and retained in the suture holders, thereby applying leverage to depressor 30 in the downward direction, at the same time they apply a lifting motion to the heart, thereby providing better visualization and access to the surgical site.

FIG. 20 shows another embodiment of the invention. This embodiment may be used with a depressor 30 such as described in connection with FIG. 19A or otherwise. Instead of a linkage approach to rib side compression and sternal lifting as described in the embodiments shown in FIGS. 16-19, the embodiment shown in FIG. 20 comprises constrained bladder(s) 252 for forming a transabdominal space. FIG. 20 shows a cross-sectional view taken when viewed from the feet toward the head of the patient, of what is at least one bladder 252 and a patient's thorax 156. A bracket 254 having a lift surface 8 and a belt 256 is also shown. Belt 256 is attached via hooks 258 to the bracket 254. Other coupling elements such as rivets, snaps, buttons, bolts or the like may be used to attached the belt 256 to the bracket 254. Thus constrained, bladder inflation causes compression of the sides of the thorax and lifting of the bracket with which there is contact. Inflation may be accomplished by air pressure, with another gas or even liquid. Any sort of automated or manual pump may be used. Inflation action is depicted by arrows. The direction of expansion may, however, be tailored or customized by varying bladder stiffness in certain sections. Preferably, the bladder(s) are made of an elastic material such as an elastomer, natural rubber, or the like, or even a non-elastic polymer such as those that are sometimes used for angioplasty balloons or the like, or other non-elastic material capable of being inflated under pressure. Any material used must of course be biocompatible and sterilizable. When not fully inflated, the kidney-shaped bladders shown may be shaped like a rectangular block or brick. The bladder shape or sized may be otherwise optimized for use.

The same is true for features of other variations of the invention as well. To the extent that built-in adjustability is not adequate to accommodate all patient or test subjects modification of features to accommodate use as described and possibly other uses is part of the present invention.

The present invention, as described above provides preferred methods and hardware for providing transabdominal access to the thoracic cavity of a patient for a number of reasons. The devices described do not require an ungainly assortment of cables bars and winches, in contrast to many instruments currently used. Rather, a compact, entirely self-contained device is provided, which is adjustable by a surgeon or other personnel from one location. The device described herein may also be manufactured with a certain amount of snap-together or clip-on parts and/or accessories that may be disposable. Still further, because a device as described above functions as a unit, it may be quickly and easily removed from the patient/operative site if emergent surgical measures are needed, or when the functions required by the device have been fully accomplished.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A transabdominal access system for a patient having a heart, a rib cage, a sternal area and an abdomen with an upper abdominal incision, comprising:
spreader portions, said spreader portions being positioned relative to each other to open the incision and produce a substernal space for access to the heart by lifting the sternal area and depressing the abdomen at the incision.

2. The system of claim 1, further comprising compression surfaces, said compression surfaces positioned across the rib cage when in use for compressing the rib cage to assist in producing the substernal space.

3. A transabdominal access system for a patient having a heart, a rib cage with opposite sides, a sternal area and an abdominal area with an upper abdominal incision, comprising:
a first spreader portion, and compression surfaces, said first spreader portion and said compression surfaces positioned relative to each other for said spreader portion to contact the sternal area and said compression surfaces to contact opposite sides of the rib cage, said system adapted to retract said sternal portion and compress said ribs to produce a substernal space for access to the heart.

4. The system of claim 3, further comprising a second spreader portion, said second spreader portion adapted to depress the abdomen at the incision.

5. The system of claim 4, wherein said first spreader portion and said second spreader portion are positioned relative to each other to open the incision and produce a substernal space for access to the heart by simultaneously lifting the sternal area and depressing the abdomen at the incision.

6. A method for providing transabdominal access for cardiac surgery in a patient having a heart, a rib cage with opposite sides, a sternal area and an abdominal area, comprising:
producing an incision in the upper abdominal area;
holding open said incision by lifting the sternal area and depressing the abdomen at the incision to produce a substernal space for access to the heart.

7. The method of claim 6, further comprising retracting the sternal area.

8. The method of claim 6, further comprising compressing the opposite sides of the rib cage to increase the substernal space for access to the heart.

9. A method for providing transabdominal access for cardiac surgery in a patient having a heart, a rib cage with opposite sides, a sternal area and an abdominal area, comprising:
producing an incision in the upper abdominal area;
holding open said incision by lifting the sternal area and compressing the opposite sides of the rib cage to produce a substernal space for access to the heart.

10. The method of claim 9, further comprising depressing the abdomen at the incision to increase the substernal space for access to the heart.

11. A transabdominal access system for a patient having an upper abdominal incision, comprising:
an upper spreader portion and a lower spreader portion, said upper spreader portion configured for lifting the sternal area of the patient at the incision and said lower spreader portion configured to depress the abdomen at the incision; and
said upper and lower spreader portions being positioned relative to each other to produce a substernal space.

12. The transabdominal access system of claim 11, wherein said lower spreader portion comprises a depressor portion, said depressor portion having a depressor surface which contacts the abdomen at the incision.

13. The transabdominal access system of claim 11, wherein said upper spreader portion comprises a lifting surface for lifting the sternal area of the patient; and
said lifting surface comprises at least one hook contacting the sternal area of the incision.

14. The transabdominal access system of claim 11, further comprising:
a first and second side portion, said first and second side portions are positioned and configured between said upper spreader portion and lower spreader portion to aid in producing a substernal space.

15. The transabdominal access system of claim 14, wherein said first and second side portions are integrally connected to said upper spreader portion and said lower spreader portion of said system.

16. The transabdominal access system of claim 14, where said first and second side portions are connected to said upper spreader portion; and
said side portions comprise a rib compression surface, said rib compression surface positioned across the rib cage when in use for compressing the rib cage to assist in producing the substernal space.

17. The transabdominal access system of claim 16, wherein said rib compression surfaces of said side portions each comprise at least one adjustable compression pad configured to contact the patient to apply an external force to the patient's rib cage.

18. The transabdominal access system of claim 17, wherein said upper spreader portion and said compression pads are configured to coordinate sternal lifting by said upper spreader portion and rib compression by said compression pads.

19. The transabdominal access system of claim 11, further comprising:
contact pads configured to stabilize said transabdominal through contact with the patient in locations aware from the substernal opening.
said upper spreader portion comprises a lifting portion for lifting and retracting the patients sternum.

20. The transabdominal access system of claim 11, wherein said upper spreader portion further comprises a retraction mechanism adapted to lift the rib cage at the site of the abdominal opening.

21. The access system of claim 21, wherein said retraction mechanism comprises a ratchet mechanism adapted to generate a force necessary to lift the sternum and maintain a lifted position of the sternum.

22. The transabdominal access system of claim 11, wherein said upper spreader portion further comprises a screw-type mechanism for lifting and retracting the sternum.

23. The transabdominal access system of claim 20, wherein said retraction mechanism comprises a piston driver.

24. The transabdominal access system of claim 20, wherein said retraction mechanism comprises a hoist.

25. The transabdominal access system of claim 20, wherein said retraction mechanism comprises a winch-type mechanism.

26. The transabdominal access system of claim 11, wherein said system is configured and mounted to a surgery table for stability of said system.

27. The transabdominal access system of claim 11, wherein said upper spreader portion further comprises a plurality of cables, said plurality of cables configured to run through the patient's chest to lift the sternal area.

28. The transabdominal access system of claim 11, wherein said lower spreader portion further comprises at least one instrument mounting element for a tool useful in surgery.

29. The transabdominal access system of claim 11, wherein said upper spreader portion comprises a lifting portion and a pair of advancement mechanisms and compression plates, said advancement mechanisms configured to adjust positions of said compression plates relative to said lifting portion.

30. The transabdominal access system of claim 29, wherein said advancement mechanisms comprise a plurality of lugs and a horizontal slider, said plurality of lugs being adjustable along said horizontal slider to optimize the placement of said compression plates relative to said lifting portion of said upper spreader portion.

31. A transabdominal access system for a patient having an upper abdominal incision, said system comprising:
a retractor portion configured to provide a tensile force to a sternal area of the patient; and
a compression portion configured to apply a compressive force to the ribs of the patient, wherein said tensile force and said compressive force are applied together to enhance a substernal opening in the patient.

32. The transabdominal access system of claim 31, wherein said retractor portion and said compression portion are linked for coordinated application of said tensile and compressive forces.

33. The transabdominal access system of claim 32, wherein said retractor portion and said compressive portion are linked by first and second linkage assemblies, said linkage assemblies configured to be symmetrical to one another.

34. The transabdominal access system of claim 33, wherein each said linkage assembly comprises a first linkage member and a second linkage member, said first linkage member and said second linkage member being pivotally coupled to form a linkage.

35. The transabdominal access system of claim 34, wherein each said linkage assembly further comprises a third linkage member, and said second linkage member has a first end coupled to said first linkage member and a second end which is pivotally coupled to said third linkage member.

36. The transabdominal access system of claim 35, wherein each said linkage assembly further comprises a forth linkage member, said forth linkage member pivotally coupled to said second linkage member.

37. The transabdominal access system of claim 36, wherein each said linkage assembly further comprises a movable lever arm which is fixedly coupled to said forth linkage member.

38. The transabdominal access system of claim 31, wherein said retractor portion comprises a main body member operatively configured to a hook member adapted to lift the sternum region of the patient.

39. The transabdominal access system of claim 33, wherein said retractor portion comprises a main body member operatively configured to a hook member adapted to lift the sternum region of the patient, wherein said compression portion includes a pair of compression pads adapted to apply said compressive force to the ribs, and wherein said main body member is pivotally coupled to both said linkage assemblies to allow for even rib compression from said compression pads and simultaneous lifting by said hook.

40. The transabdominal access system of claim 36, wherein said retractor portion comprises a main body member operatively configured to a hook member adapted to lift the sternum region of the patient, wherein said forth linkage member is pivotally coupled to said main body member in a fixed location; and wherein lifting or lowering of said main body member allows the coordinated movement of said third and forth linkage members.

41. The transabdominal access system of claim 39, further comprising a rack-type mechanism operatively connected to said main body and said linkage assemblies for adjusting rib compression and sternal lifting.

42. The transabdominal access system of claim 41, wherein said rack-type mechanism comprises a rack, a lever arm, a first side and a second side, where said first side is configured to be movable over said rack relative to said second side; and said lever arm is configured to lock said first and said second side of said rack-type mechanism into a desired position.

43. The transabdominal access system of claim 31, further comprising a depressor mechanism configured to depress an abdominal area of a patient, thereby further enhancing the substernal opening.
